(19) United States
(12) Patent Application Publication
(10) Pub. No.: US 2023/0248788 A1
(43) Pub. Date: Aug. 10, 2023

(54) COMPOSITIONS AND RELATED METHODS FOR SUPPORTING COMPANION ANIMALS WITH GASTROINTESTINAL DISORDERS

(71) Applicant: CANBIOCIN INC., Edmonton (CA)

(72) Inventors: Qixing Ou, Edmonton (CA); John F. Burlet, Edmonton (CA); Petya Koleva, Edmonton (CA)

(73) Assignee: CANBIOCIN INC., Edmonton, AB (CA)

(21) Appl. No.: 18/004,672

(22) PCT Filed: Jun. 29, 2021
(86) PCT No.: PCT/CA2021/050889

§ 371 Date: Jan. 7, 2023

Related U.S. Application Data
(60) Provisional application No. 63/045,283, filed on Jun. 29, 2020.

(51) Int. Cl. A61K 35/747; A61K 35/744; A61K 47/12; A61K 47/36; A61P 1/14

(52) U.S. Cl. CPC A61K 35/747; A61K 35/744; A61K 47/12; A61K 47/36; A61P 1/14

(57) ABSTRACT

Compositions are provided for providing support to companion animals affected by Inflammatory Bowel Disease (IBD) and/or Irritable Bowel Syndrome (IBS). In some embodiments, the composition comprises at least one isolated strain of wolf probiotic bacteria and at least one isolated strain of canine probiotic bacteria. In some embodiments, the composition further comprises at least one prebiotic. Also provided are related methods for preparing a composition and for treating IBS and/or IBD in a subject.

### RELATED APPLICATION

The present disclosure claims priority to U.S. Provisional Patent Application No. 63/045,283, filed Jun. 29, 2020, the entire contents of which are incorporated by reference herein.

### TECHNICAL FIELD

The present disclosure relates to compositions for treating gastrointestinal disorders. More particularly, the present disclosure relates to compositions and related methods for supporting companion animals affected by Inflammatory Bowel Disease (IBD) and Irritable Bowel Syndrome (IBS) in companion animals.

### BACKGROUND

Animals with Inflammatory Bowel Disease (IBD) or Irritable Bowel Syndrome (IBS) commonly present with symptoms including but not limited to: diarrhoea, abdominal pain, accelerated gastrointestinal transit time, and altered diet preference. The common implicating features include genetic predispositions, impaired gut barrier function, and altered gut microbiota. Possible therapeutic methods include the application of antibiotics, probiotics, prebiotics, and faecal transplantation (Major & Spiller, 2014).

Although a variety of therapies have been developed for treating IBD and IBS in humans, such treatments are generally not effective in animals. Few treatments are commercially available that are optimized for treatment of IBS and IBD in companion animals such as domestic dogs.

### SUMMARY

In one aspect, there is provided a composition comprising: a first isolated strain of wolf probiotic bacteria, wherein the first isolated strain of wolf probiotic bacteria is a species of the Lactobacillaceae family; a second isolated strain of wolf probiotic bacteria, wherein the second isolated strain of wolf probiotic bacteria is a species of the Enterococcaceae family; and at least one isolated strain of canine probiotic bacteria, wherein the at least one isolated strain of canine probiotic bacteria comprises at least one species of the Lactobacillaceae family.

In some embodiments, the composition further comprises at least one prebiotic.

In some embodiments, the at least one prebiotic comprises at least one of maltodextrin, humic acid, and fulvic acid.

In some embodiments, the first isolated strain of wolf probiotic bacteria is a Levilactobacillus species and the second isolated strain of wolf probiotic bacteria is an Enterococcus species.

In some embodiments, the first isolated strain of wolf probiotic bacteria is Levilactobacillus brevis and the second isolated strain of wolf probiotic bacteria is Enterococcus faecium.

In some embodiments, the first isolated strain of wolf probiotic bacteria is Levilactobacillus brevis WF-1B IDAC Accession number 051120-02 or a mutant strain thereof; and wherein the second isolated strain of wolf probiotic bacteria is Enterococcus faecium strain WF-3 IDAC Accession number 181218-03 or a mutant strain thereof.

In some embodiments, the at least one isolated strain of canine probiotic bacteria comprises a Lacticaseibacillus species and a Limosilactobacillus species.

In some embodiments, the at least one strain of canine probiotic bacteria comprises Lacticaseibacillus casei and Limosilactobacillus fermentum.

In some embodiments, the at least one isolated strain of canine probiotic bacteria comprises: Lacticaseibacillus casei strain K9-1 IDAC Accession number 210415-01 or a mutant strain thereof; and Limosilactobacillus fermentum strain K9-2 IDAC Accession number 210415-02 or a mutant strain thereof.

In some embodiments, the composition comprises: Levilactobacillus brevis strain WF-1B IDAC Accession number 051120-02; Enterococcus faecium strain WF-3 IDAC Accession number 181218-03; Lacticaseibacillus casei strain K9-1 IDAC Accession number 210415-01; Limosilactobacillus fermentum strain K9-2 IDAC Accession number 210415-02; at least one of maltodextrin, humic acid, and fulvic acid.

In another aspect, there is provided a use of the composition of any one of claims 1 to 10 to treat Inflammatory Bowel Disease (IBD) and/or Irritable Bowel Syndrome (IBS) in a subject.

In another aspect, there is provided a method for treating IBD and/or IBS in a subject comprising administering the composition of any one of claims 1 to 10 to the subject.

In some embodiments, the subject is a domestic dog.

In some embodiments, the composition is administered orally.

In another aspect, there is provided a kit comprising the composition of any one of claims 1 to 10 in a container and instructions for administration of the composition to treat IBD and/or IBS.

In another aspect, there is provided a method for making a composition for treating IBD and/or IBS, comprising: providing a first isolated strain of wolf probiotic bacteria, wherein the first isolated strain of wolf probiotic bacteria is a species of the Lactobacillaceae family; providing a second isolated strain of wolf probiotic bacteria, wherein the second isolated strain of wolf probiotic bacteria is a species of the Enterococcaceae family; providing at least one isolated strain of canine probiotic bacteria, wherein the at least one isolated strain of canine probiotic bacteria comprises at least one species of the Lactobacillaceae family; and combining the first and second isolated strains of wolf probiotic bacteria and the at least one strain of canine probiotic bacteria.

In some embodiments, the method further comprises providing at least one prebiotic and combining the at least one prebiotic with the first and second isolated strains of wolf probiotic bacteria and the at least one isolated strain of canine probiotic bacteria.

In another aspect, there is provided Levilactobacillus brevis WF-1B IDAC Accession number 051120-02.

In another aspect, there is provided a composition comprising Levilactobacillus brevis WF-1B IDAC Accession number 051120-02 or a mutant strain thereof and at least one additional ingredient.

In another aspect, there is provided a use of Levilactobacillus brevis WF-1B IDAC Accession number 051120-02 or a mutant strain thereof in the preparation of a medicament for treating or preventing intestinal dysbiosis in a subject.

In another aspect, there is provided a method for treating or preventing intestinal dysbiosis in a subject comprising administering Levilactobacillus brevis WF-1B IDAC Accession number 051120-02 or a mutant strain thereof to a subject.

Other aspects and features of the present disclosure will become apparent, to those ordinarily skilled in the art, upon review of the following description of the specific embodiments of the disclosure.

### BRIEF DESCRIPTION OF THE DRAWINGS

Some aspects of the disclosure will now be described in greater detail with reference to the accompanying drawings. In the drawings:

FIG. 1A shows a 16S rDNA sequence of Limosilactobacillus reuteri WF-1 (SEQ. ID NO: 1); FIG. 1B shows a 16S rDNA sequence of Ligilactobacillus animalis WF-2 (SEQ. ID NO: 2); FIG. 1C shows a 16S rDNA sequence of Enterococcus faecium WF-3 (SEQ. ID NO: 3); FIG. 1D shows a 16S rDNA sequence of Lactiplantibacillus plantarum WF-4 (SEQ. ID NO: 4); FIG. 1E shows a 16S rDNA sequence of L. brevis WF-5 (SEQ. ID NO: 5); FIG. 1F shows a 16S rDNA sequence of Latilactobacillus curvatus WF-6 (SEQ. ID NO: 6); FIG. 1G shows a 16S rDNA sequence of L. reuteri WF-7 (SEQ. ID NO: 7);

FIG. 2 shows a 16S rDNA sequence of L. brevis WF-1B (SEQ ID NO: 10);

FIG. 3A shows a 16S rDNA sequence of L. casei K9-1 (SEQ. ID NO: 8);

FIG. 3B shows a 16S rDNA sequence of L. fermentum K9-2 (SEQ. ID NO: 9);

FIG. 4 is a flowchart of a method for preparing a composition, according to some embodiments;

FIG. 5 is a photo of Gram staining results showing the bacterial morphology of L. brevis WF-1B; 1B;

FIG. 6 is a graph showing the auto-aggregation results for L. brevis WF-1B;

FIG. 7 is a graph showing cell surface hydrophobicity assay results for L. brevis WF-1B;

FIG. 8 is a graph showing low pH tolerance assay results for L. brevis WF-1B;

FIG. 9 is a graph showing bile salt tolerance assay results for L. brevis WF-1B;

FIG. 10 is a graph showing gastric digestive enzyme (3.2 mg/mL pepsin) tolerance assay results for L. brevis WF-1B;

FIG. 11 is a graph showing intestinal digestive enzyme (10 mg/mL pancreatin) tolerance assay results for L. brevis WF-1B;

FIG. 12 is a graph showing cell binding assay results for L. brevis WF-1B;

FIG. 13 is a set of graphs showing relative abundance of specific bacterial groups/species in fecal samples collected on Day −1 (pre-treatment) and Day 19 (during treatment) from control (fed dogs with placebos; black bar) and test (fed dogs with probiotics; white bar) groups (panel A=total bacteria; panel B=Lactobacillus spp.; panel C=Enterococcus spp.; panel D=L. casei; panel E=L. fermentum; panel F=L. brevis; panel G=E. faecium); vertical bars represent means±SEM; asterisk (*) indicates the two sets of data are statistically significant (P<0.10); any two sets of data without a common superscript indicate they are statistically significantly different (P<0.05);

FIG. 14 is a graph showing quantification of total short-chain fatty acids (SCFAs) present in fecal samples collected on Day −1 and Day 19 from control (black bar) and test (white bar) groups (vertical bars represent means±SEM); and

FIG. 15 is a set of graphs showing quantification SCFAs present in fecal samples collected on Day −1 (white bars) and Day 19 (grey bars) from control (panels A and B) and test (panels C and D) groups (vertical bars represent means±SEM).

### DETAILED DESCRIPTION

Generally, the present disclosure provides a composition comprising at least one isolated strain of wolf (Canis lupus) probiotic bacteria and at least one isolated strain of canine (C. l. familiaris) probiotic bacteria. In some embodiments, the composition further comprises at least one prebiotic. Also provided is a related method for preparing a composition and a method for treating IBS and/or IBD in a subject.

The composition may be a synbiotic composition. As used herein, “synbiotic” refers to a composition that comprises at least one probiotic component and at least one prebiotic component. As used herein, “probiotic” refers to a microbial cell culture or preparation that has at least one beneficial effect on a host organism. The beneficial effects on the host organism may include, for example, a beneficial effect on the at least one of the host's digestive system, immune system, and brain-gut-microbiome system. As used herein, “prebiotic” refers to a substance that supports the growth and/or activity of at least one beneficial micro-organism.

As used herein, “isolated” or “isolate”, when used in reference to a strain of bacteria, refers to bacteria that have been separated from their natural environment. In some embodiments, the isolated strain or isolate is a biologically pure culture of a specific strain of bacteria. As used herein, “biologically pure” refers to a culture that is substantially free of other strains of organisms.

The composition may comprise at least one isolated strain of wolf probiotic bacteria. As used herein “wolf probiotic bacteria” refers to bacteria with probiotic activity isolated from a wolf. As used herein, “wolf” refers to an animal of the Canis lupus species, including any known subspecies, with the exception of Canis lupus familiaris. A wolf may also be known as a gray wolf, grey wolf, timber wolf, or tundra wolf. In some embodiments, the wolf is a free-ranging wolf. In some embodiments, the wolf is a free-ranging wolf native to Prince Albert National Park in Saskatchewan, Canada.

Each isolated strain of wolf probiotic bacteria may be an isolated strain of gastrointestinal bacteria native to the gastrointestinal tract of a wolf. In some embodiments, the isolated strain(s) are isolated from wolf feces. In other embodiments, each isolated strain may be isolated from a wolf by any other suitable means.

In some embodiments, at least one isolated strain is a strain of lactic acid bacteria. In some embodiments, at least one isolated strain is a species of the Lactobacillaceae family including, but not limited to, a species of the Limosilactobacillus, Ligilactobacillus, Lactiplantibacillus, Levilactobacillus, or Latilactobacillus genera or any other species of the former Lactobacillus genus (also referred to as “lactobacilli”). In some embodiments, at least one isolated strain is a species of the Enterococcaceae family including, for example, a species of the Enterococcus genus. In other embodiments, the isolated strain is any other genus of gastrointestinal bacteria native to a wolf gastrointestinal tract.

In some embodiments, at least one isolated strain of wolf probiotic bacteria is selected from Limosilactobacillus reuteri, (formerly Lactobacillus reuteri), Ligilactobacillus animalis (formerly Lactobacillus animalis), Enterococcus faecium, Lactiplantibacillus plantarum (formerly Lactobacillus plantarum), Levilactobacillus brevis (formerly Lactobacillus brevis), and Latilactobacillus curvatus (formerly Lactobacillus curvatus). A person skilled in the art will understand that the current and former names refer to the same species and embodiments are not limited to any one specific terminology.

In some embodiments, at least one isolated strain is selected from the strains listed in Table 1 below and disclosed in international PCT (Patent Cooperation Treaty) patent application PCT/CA2019/051140, published as WO2020/037414, incorporated herein by reference. For each bacterial strain in Table 1, a biologically pure stock of each isolate was deposited in the International Depositary Authority of Canada (IDAC) (1015 Arlington Street, Winnipeg, Manitoba, Canada R3E 3R2) under the Budapest Treaty on Dec. 18, 2018.

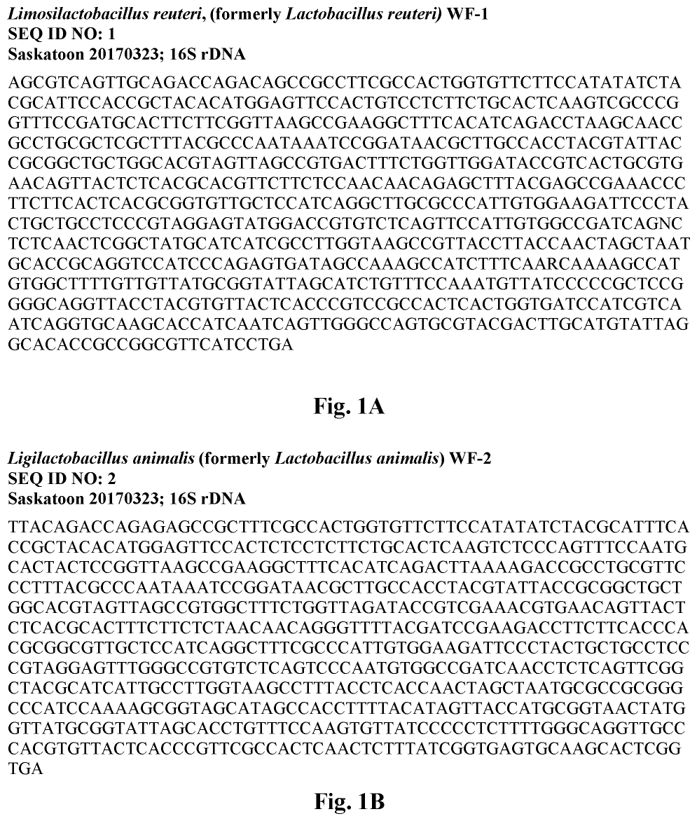

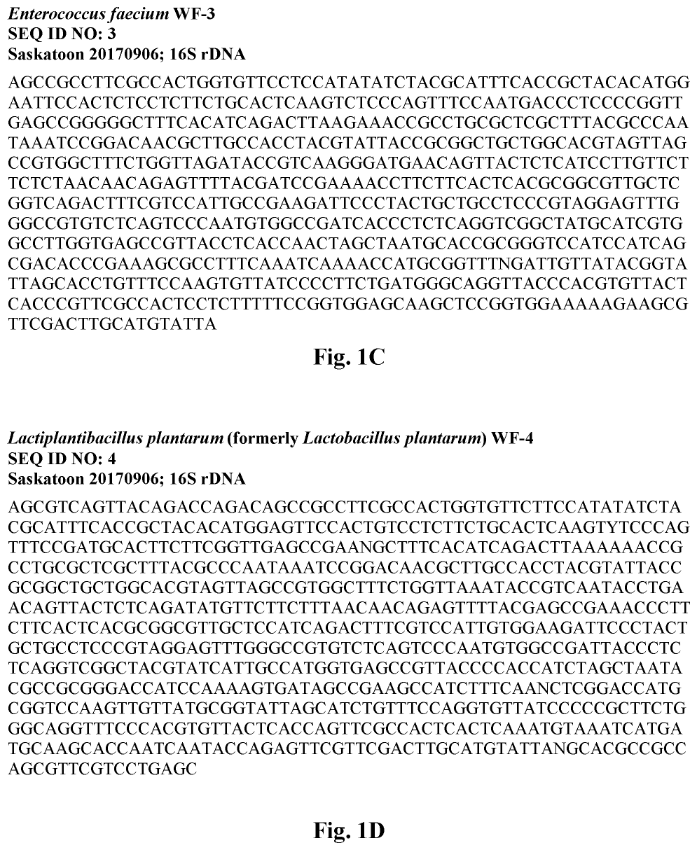

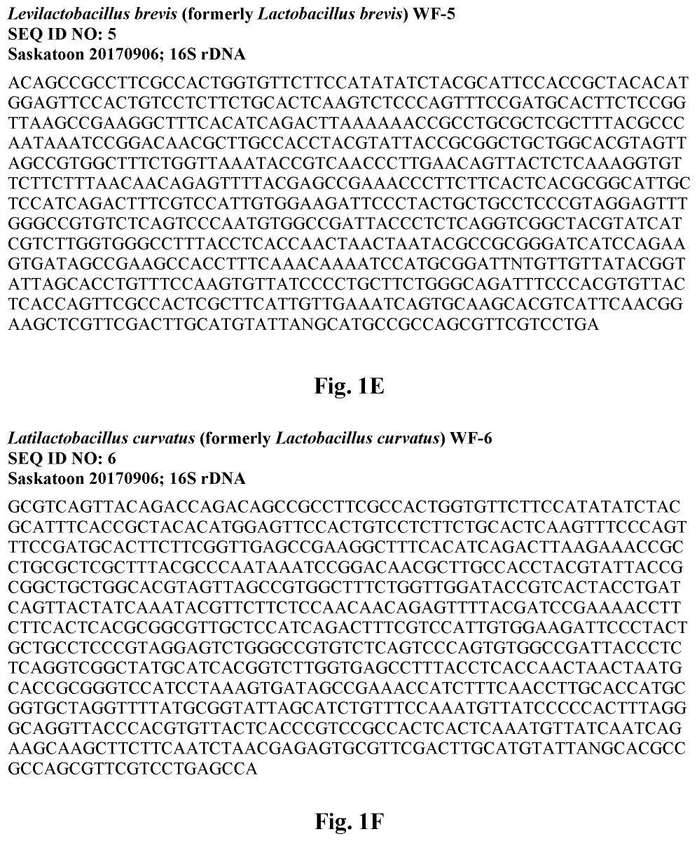

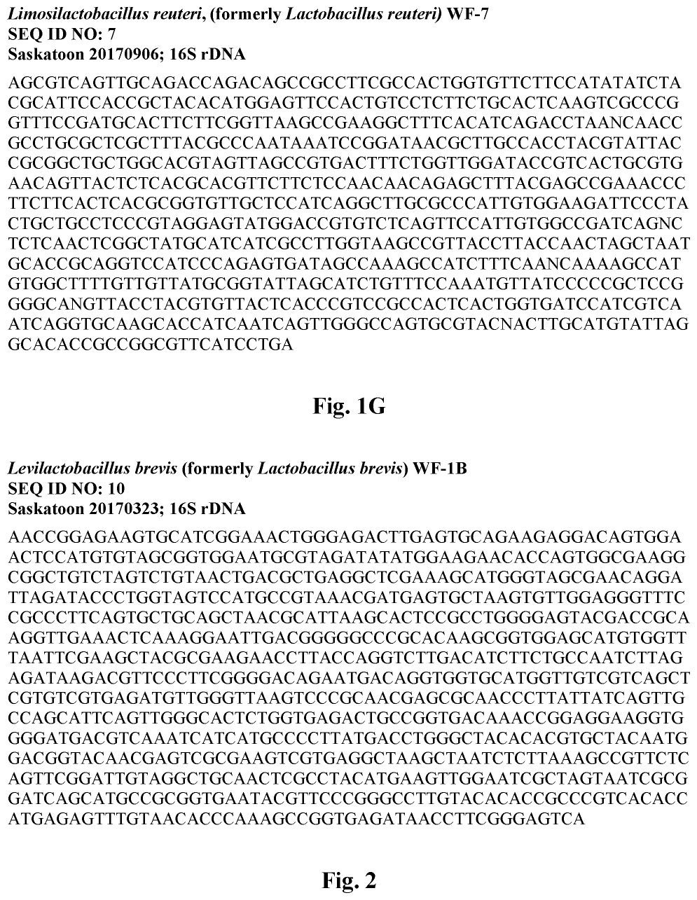

TABLE 1

| | IDAC | | Figure Showing |
|---|---|---|---|
| | Accession | 16S rDNA | 16S rDNA |
| Strain | Number | Sequence | Sequence |
| Limosilactobacillus | 181218-01 | SEQ. ID NO: 1 | FIG. 1A |
| reuteri WF-1 | | | |
| Ligilactobacillus | 181218-02 | SEQ. ID NO: 2 | FIG. 1B |
| animalis WF-2 | | | |
| Enterococcus faecium | 181218-03 | SEQ. ID NO: 3 | FIG. 1C |
| WF-3 | | | |
| Lactiplantibacillus | 181218-04 | SEQ. ID NO: 4 | FIG. 1D |
| plantarum WF-4 | | | |
| Levilactobacillus | 181218-05 | SEQ. ID NO: 5 | FIG. 1E |
| brevis WF-5 | | | |
| Latilactobacillus | 181218-06 | SEQ. ID NO: 6 | FIG. 1F |
| curvatus WF-6 | | | |
| Limosilactobacillus | 181218-07 | SEQ. ID NO: 7 | FIG. 1G |
| reuteri WF-7 | | | |

In some embodiments, a 16S ribosomal DNA (rDNA) sequence can be used to identify genus and species of bacteria. Sequencing of 16S rDNA sequences may be performed using the methods described in PCT/CA2019/051140. The partial 16S rDNA sequences of the isolated strains listed in Table 1 are shown in FIGS. 1A to 1G.

In some embodiments, one of the isolated strains is Levilactobacillus brevis WF-1B, isolated from the feces of a free-ranging wolf native to Prince Albert National Park in Saskatchewan, Canada. A biologically pure stock of L. brevis WF-1B was deposited in the International Depositary Authority of Canada (IDAC) (1015 Arlington Street, Winnipeg, Manitoba, Canada R3E 3R2) under the Budapest Treaty on Nov. 5, 2020 and assigned accession number 051120-02. The partial 16S rDNA sequence of L. brevis WF-1B is shown in FIG. 2 (SEQ. ID NO: 10).

As demonstrated in the Examples below, the bacteria of L. brevis WF-1B show tolerance to low pH and the presence of bile salts. The bacteria also show tolerance to the presence of at least one gastric and/or intestinal digestive enzyme. These results indicate that L. brevis WF-1B is capable of surviving passage through the acidic canine stomach and through the canine intestine. As used herein, “survive” means that the viable cell count of a test culture (as measured in colony forming units (CFU) per mL) is above detection limit [1.7 log₁₀(CFU/mL) or 50 CFU/m L].

The Examples below also show that the bacteria of L. brevis WF-1B have autoaggregation ability and cell surface hydrophobicity, indicating that the bacterial cells may be able to bind host intestinal epithelial cells in the subject to facilitate colonization of the gastrointestinal tract. The bacteria of L. brevis WF-1B were also found to bind canine epithelial cells in vitro.

The bacteria of L. brevis WF-1B have also been shown to produce inhibitory substances to inhibit the growth of at least one pathogenic or spoilage microorganism. As discussed below, WF-1B was found to inhibit several strains of pathogenic or spoilage microorganisms including Escherichia coli, Salmonella enterica, Listeria monocytogenes, Staphylococcus aureus, and Enterococcus faecalis.

L. brevis WF-1B is susceptible to gentamicin, streptomycin, and erythromycin, but resistant to ampicillin, kanamycin, clindamycin, tetracycline, and chloramphenicol. Antibiotic susceptibility may be desirable to prevent the transfer of antibiotic resistance genes to other bacteria, including pathogenic bacteria. The lowest antibiotic concentration for which no bacteria growth is observed is referred to as the minimum inhibitory concentration (MIC). In some embodiments, L. brevis WF-1B has an MIC value for at least one antibiotic that is at or below the MIC cut off value set by the European Food Safety Authority (EFSA). Whole genome sequence analysis shows that the resistance of L. brevis WF-1B to ampicillin, clindamycin, tetracycline, and chloramphenicol is classified as either intrinsic resistance or acquired resistance due to genomic mutation. The risk of horizontal antibiotic resistance (AR) gene transfer is low. Therefore, it is considered safe to use L. brevis WF-1B as feed additives in animal nutrition.

In some embodiments, L. brevis WF-1B displays one or more other desirable properties and such properties are not limited to only those described herein.

In some embodiments, the composition comprises a mutant of one of the strains described above. As used herein, a “mutant” or a “mutant strain” refers to a bacterial strain that has at least 95% homology, at least 96% homology, at least 97% homology, at least 98% homology, at least 99% homology, or at least 99.5% homology to the 16S rDNA sequence of a reference bacterial strain but that otherwise has one or more DNA mutations in one or more other DNA sequences in the bacterial genome. DNA mutations may include base substitutions including transitions and transversions, deletions, insertions, and any other type of natural or induced DNA modification.

In some embodiments, the composition comprises a combination of isolated strains of wolf probiotic bacteria. In some embodiments, the composition comprises a first isolated strain of wolf probiotic bacteria and a second isolated strain of wolf probiotic bacteria. In some embodiments, the first isolated strain is a species of the Lactobacillaceae family and the second isolated strain is a species of the Enterococcaceae family.

The first isolated strain may comprise, for example, an isolated strain of the Limosilactobacillus, Ligilactobacillus, Lactiplantibacillus, Levilactobacillus, or Latilactobacillus genera (or any other species of the former Lactobacillus genus). In some embodiments, the first isolated strain is a Levilactobacillus species such as Levilactobacillus brevis. In some preferred embodiments, the first isolated strain is Levilactobacillus brevis WF-1B IDAC Accession number 051120-02 or a mutant strain thereof.

The second isolated strain may comprise, for example, an isolated strain of the Enterococcus genus. In some embodiments, the second isolated strain is Enterococcus faecium. In some preferred embodiments, the second isolated strain is Enterococcus faecium strain WF-3 IDAC Accession number 181218-03 or a mutant strain thereof.

In some embodiments, the composition may further comprise additional isolated strains of wolf probiotic bacteria such as a third, fourth, fifth isolated strain, etc. In other embodiments, the composition may comprise any other suitable combination of isolated strains of wolf probiotic bacteria.

The composition may further comprise at least one isolated strain of canine probiotic bacteria. As used herein, “canine probiotic bacteria” or “dog probiotic bacteria” refers to bacteria with probiotic activity isolated from a dog. As used herein, “dog” or “domestic dog” refers to an animal of the Canis lupus familiaris subspecies. Some taxonomic authorities alternatively recognize domestic dogs as a distinct species Canis familiaris.

Each isolated strain of canine probiotic bacteria may be an isolated strain of gastrointestinal bacteria native to the gastrointestinal tract of a dog. In some embodiments, the isolated strain(s) are isolated from dog feces. In other embodiments, each isolated strain may be isolated from a dog by any other suitable means.

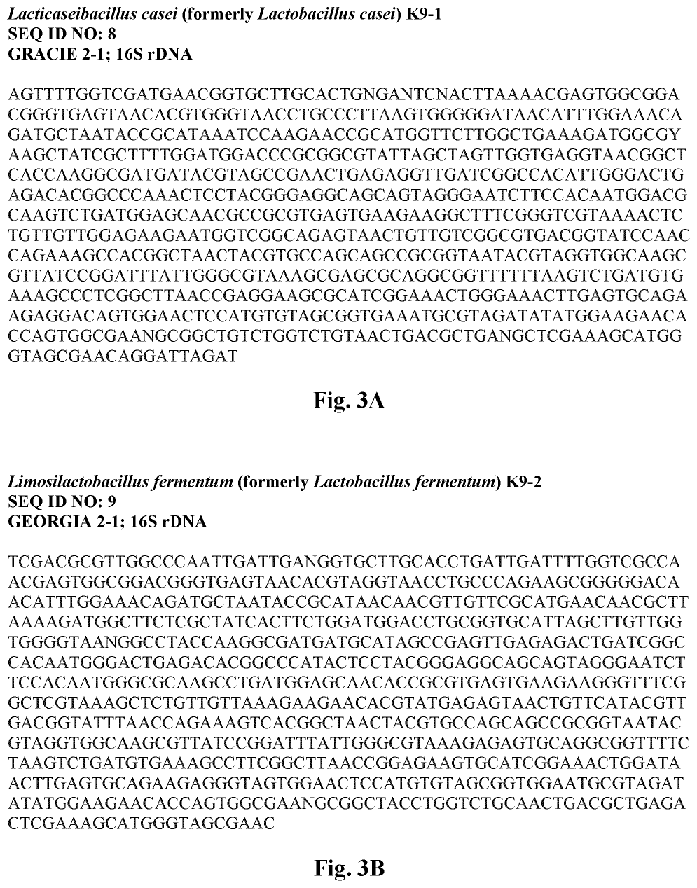

In some embodiments, at least one isolated strain of canine probiotic bacteria is a strain of lactic acid bacteria. In some embodiments, at least one isolated strain is a species of the Lactobacillaceae family including, but not limited to, a species of the Limosilactobacillus or Lacticaseibacillus genera (or any other species of the former Lactobacillus genus). In some embodiments, at least one isolated strain is selected from Lacticaseibacillus casei (formerly Lactobacillus casei) or Limosilactobacillus fermentum (formerly Lactobacillus fermentum). In some embodiments, at least one isolated strain is selected from the strains listed in Table 2 and disclosed in Canadian Patent No. 2,890,965, incorporated herein by reference. For each bacterial strain in Table 2, a biologically pure stock of each isolate was deposited in the International Depositary Authority of Canada (IDAC) (1015 Arlington Street, Winnipeg, Manitoba, Canada R3E 3R2) under the Budapest Treaty on Apr. 21, 2015. The partial 16S rDNA sequences of the strains in Table 2 are shown in FIGS. 3A and 3B.

TABLE 2

| | IDAC | | Figure Showing |
|---|---|---|---|
| | Accession | 16S rDNA | 16S rDNA |
| Strain | Number | Sequence | Sequence |
| Lacticaseibacillus | 210415-01 | SEQ. ID NO: 8 | FIG. 2A |
| casei K9-1 | | | |
| Limosilactobacillus | 210415-02 | SEQ. ID NO: 9 | FIG. 2B |
| fermentum K9-2 | | | |

In some embodiments, at least one isolated strain is a mutant of one of the strains listed in Table 2.

The composition may comprise a combination of isolated strains of canine probiotic bacteria. In some embodiments, the composition comprises a first isolated strain of canine probiotic bacteria and a second isolated strain of canine probiotic bacteria. The first and second strains may both be species of the Lactobacillaceae family. In some embodiments, the first isolated strain is a Lacticaseibacillus species, such as Lacticaseibacillus casei, and the second isolated strain is a Limosilactobacillus species, such as Limosilactobacillus fermentum. In some preferred embodiments, the composition comprises Lacticaseibacillus casei K9-1 IDAC Accession number 210415-01 and Limosilactobacillus fermentum strain K9-2 IDAC Accession number 210415-02.

In some embodiments, the composition may further comprise additional isolated strains of canine probiotic bacteria such as a third, fourth, fifth isolated strain, etc. In other embodiments, the composition may comprise any other suitable combination of isolated strains of canine probiotic bacteria.

As demonstrated in the Examples below, the isolated strains of wolf probiotic bacteria and canine probiotic bacteria are generally well tolerated when administrated orally to domestic dogs. The isolated strains are also capable of surviving the passage through the canine gastrointestinal tract. In some embodiments, each isolated strain has one or more beneficial physiological effects on a subject, as described in more detail below.

In some embodiments, the isolated strains of wolf probiotic bacteria and canine probiotic bacteria may be in a viable form. In some embodiments, the isolated strains may be in a lyophilized (freeze-dried) form. In other embodiments, the isolated strains are in the form of a liquid suspension.

In some embodiments, the composition is a synbiotic composition further comprising at least one prebiotic. In some embodiments, the prebiotic comprises a polysaccharide prebiotic. For example, the prebiotic may comprise maltodextrin. In other embodiments, the prebiotic comprises at least one humus substance component, including humic acid and/or fulvic acid. The terms “humic acid” and “fulvic acid” will be understood to include heterogeneous mixtures of humic acids and fulvic acids, respectively, as well as any salts, esters, or other derivatives thereof. Humic acids are generally water soluble at alkaline pH but become less soluble under acidic conditions, whereas fulvic acids are generally water soluble at all pH values.

In some embodiments, the composition comprises a combination of two or more prebiotics. For example, the composition may comprise a combination of maltodextrin and humic and/or fulvic acids. In other embodiments, the composition may comprise any other suitable prebiotic or combination of prebiotics. The prebiotic component of the composition may be in a liquid form, powder form, or any one suitable form.

In some embodiments, at least one prebiotic may support the growth and/or activity of the wolf probiotic bacteria and/or canine probiotic bacteria in the composition. In some embodiments, at least one prebiotic may have one or more beneficial physiological effects on a subject, as described in more detail below.

As one specific example, the composition may be a synbiotic composition comprising: Levilactobacillus brevis WF-1B IDAC Accession number 051120-02; Enterococcus faecium strain WF-3 IDAC Accession number 181218-03; Lacticaseibacillus casei strain K9-1 IDAC Accession number 210415-01; Limosilactobacillus fermentum strain K9-2 IDAC Accession number 210415-02; and at least one of maltodextrin, humic acid, and fulvic acid.

In some embodiments, the composition comprises each of the isolated strains in equal proportion, for example, by cell count or by optical density. In other embodiments, the composition may comprise the isolated strains in any other suitable proportion. In some embodiments, the composition comprises at least about 1×10⁷ CFU/g of each isolated strain. In some embodiments, the composition comprises between about 1×10⁷ CFU/g and about 1×10¹¹ CFU/g.

In some embodiments, the composition comprises at least about 1 mg/mL prebiotic or between about 1 mg/mL and about 20 mg/mL, or between about 5 mg/mL and about 15 mg/mL prebiotic. In some embodiments, the composition comprises approximately 10 mg/mL maltodextrin or approximately 10 mg/mL humic acid and/or fulvic acid. In other embodiments, the composition comprises any other suitable concentration of maltodextrin, humic acid and/or fulvic acid.

In some embodiments, the composition comprises a synergistically effective amount of at least one isolated strain of wolf probiotic bacteria; a synergistically effective amount of at least one isolated strain of canine probiotic bacteria; and/or a synergistically effective amount of at least one prebiotic. As used herein, “synergistically effective amount” refers to an amount of one component sufficient to elicit a synergistic effect with at least one other component in the composition.

The composition can be an immediate-, fast-, slow-, sustained-, or delayed-release composition or any other suitable type of composition.

In some embodiments, the composition may further comprise at least one pharmaceutically or nutritionally acceptable excipient. Non-limiting examples of suitable excipients include fillers, binders, carriers, diluents, stabilizers, lubricants, glidants, coloring agents, flavoring agents, coatings, disintegrants, preservatives, sorbents, sweeteners and any other pharmaceutically or nutritionally acceptable excipient.

In some embodiments, the composition may further comprise at least one encapsulation material. Non-limiting examples of suitable encapsulation materials include polysaccharides such as alginate, plant/microbial gums, chitosan, starch, k-carrageenan, cellulose acetate phthalate; proteins such as gelatin or milk proteins; fats; and any other suitable encapsulation material. The isolated strains may be encapsulated in the encapsulated material by spray drying, extrusion, gelation, droplet extrusion, emulsion, freeze-drying, or any other suitable encapsulation method. Encapsulation of the bacterial cells of the isolated strains may protect the cells and extend the shelf-life of the composition.

In some embodiments, the composition may further comprise at least one additional pharmaceutical or nutritional ingredient. Non-limiting examples of additional ingredients include: at least one vitamin, mineral, fiber, fatty acid, amino acid, or any other suitable pharmaceutical or nutritional ingredient.

In some embodiments, the composition is an ingestible composition. As used herein, “ingestible” refers to a substance that is orally consumable by the subject.

In some embodiments, the ingestible composition is in the form of a dietary supplement. The dietary supplement may be in the form of a powder, a capsule, a gel capsule, a microcapsule, a bead, a tablet, a chewable tablet, a gummy, a liquid, or any other suitable form of dietary supplement.

In some embodiments, the ingestible composition is in the form of a food product. In some embodiments, the food product is in any form suitable for a companion animal, particularly a domestic dog. In some embodiments, the food product is a solid food product. In some embodiments, the solid food product may be dry, wet, semi-moist, frozen, dehydrated, freeze-dried, or in any other suitable form. Examples of suitable solid food products include but are not limited to dog foods such as kibble, biscuits, chews, wet dog food, raw dog food including raw meat, freeze-dried yogurt, and others. In some embodiments, the solid food product may in the form of a dog treat including, for example, a freeze-dried dog treat.

In some embodiments, the solid food product is formulated with the composition therein. In other embodiments, the composition may be added to the solid food product post-production.

In some embodiments, the ingestible composition may be in the form of a surface coating for a solid food product. In some embodiments, the surface coating comprises a carrier to allow the bacteria to adhere to the surface of the solid food product. The carrier may be, for example, an edible oil or any other suitable carrier. As one example, an oil-based surface coating can be applied to kibbled dog food post-production and post-cooling.

In other embodiments, the ingestible composition may be provided in a powder form suitable to sprinkle onto the surface of the solid food product. In other embodiments, the ingestible composition may be provided in a liquid form to spray, pour, or drop onto the surface of the solid food product.

In other embodiments, the food product is a liquid food product. Non-limiting examples of liquid food products include beverages, broths, oil suspensions, gravies, milk-based products, liquid or semi-solid yogurt, and others.

In some embodiments, the liquid food product is formulated with the composition therein. In other embodiments, the composition may be added to the liquid food product post-production. In some embodiments, the ingestible composition may be provided in a powder form and the powder may be dissolved in water, milk, or any other suitable liquid to form the liquid food product. In other embodiments, the ingestible composition may be provided in a liquid form and may be mixed with water, milk, or any other suitable liquid to form the liquid food product. Alternatively, the liquid food product may be sprayed, poured, or dropped directly into the subject's mouth.

In other embodiments, the ingestible composition may be in any other form suitable for ingestion by a companion animal, particularly a domestic dog. In other embodiments, the composition may be in a non-ingestible form, for example, as a suppository, or any other suitable form.

Provided herein is a method for treating a gastrointestinal disorder in a subject with the composition described above. Also provided herein is a use of the composition for treating a gastrointestinal disorder in subject. As used herein, “treat” or “treatment” refers to obtaining a desired pharmacologic and/or physiologic effect. The effect can be prophylactic in terms of completely or partially preventing a health condition or symptom thereof and/or can be therapeutic in terms of completely or partially ameliorating at least one symptom of a health condition and/or adverse effect attributable to the health condition. For greater clarity, it will be understood that the terms “treat” or “treatment” in this context are intended to include providing any beneficial physiological effect to a subject and their meaning is not limited to preventing or curing a specific disorder or health condition.

In some embodiments, the subject is a companion animal including but not limited to a domestic dog. In some embodiments, the dog is an adult dog. In other embodiments, the dog is at any other stage of development.

In some embodiments, the composition may be used to treat Inflammatory Bowel Disease (IBD) and/or Irritable Bowel Syndrome (IBS) in the subject. As used herein, “IBD” refers to an inflammatory condition of the gastrointestinal tract including, for example, Crohn's disease and ulcerative colitis. As used herein, “IBS” refers to a functional bowel disorder in which the subject experiences recurrent or chronic gastrointestinal symptoms. Common symptoms include, but are not limited to: diarrhoea, abdominal pain, accelerated gastrointestinal transit time, and altered diet preference. In some embodiments, the composition may be used to treat one or more of the symptoms of IBD and/or IBS. In other embodiments, the composition may be used to treat other gastrointestinal disorders including, for example, other functional bowel disorders.

Without being limited by theory, it is believed that the combination of isolated strains of wolf probiotic bacteria and dog probiotic bacteria, along a prebiotic component, act synergistically to induce at least one beneficial physiological effect to ameliorate the discomfort associated with IBD and/or IBS in the subject.

Gastrointestinal disorders such as IBD and IBS are associated with local intestinal inflammation and loss of the integrity of the intestinal barrier. In some embodiments, the beneficial physiological effects of the composition include positive effects on gut tight junction protein function and restoring or preventing barrier disturbances of the intestinal tissue. In some embodiments, the beneficial physiological effects also include helping to maintain intestinal tissue viability. The composition may also reduce the expression of pro-inflammatory cytokines in the intestine including, for example, TNF-α.

In addition, IBD and IBS are also associated with altered intestinal microbiota and reduced levels of short chain fatty acids (SCFAs), which are produced by fermentation of fibers by intestinal bacteria. SCFAs are important metabolites in maintaining intestinal homeostasis. In some embodiments, the beneficial physiological effects of the composition include positive effects on the constitution of the intestinal microbiota and/or the production of SCFAs, such as increased levels of acetate, propionate and/or butyrate.

In some embodiments, the composition provides one or more additional beneficial physiological effects and embodiments are not limited to only the benefits disclosed herein.

In some embodiments, the isolated strains of wolf and dog probiotic bacteria and the prebiotic component may all contribute to one or more of the same beneficial physiological effects. Alternatively (or additionally), the wolf probiotic bacteria, dog probiotic bacteria, and/or the prebiotic component may contribute to one or more different beneficial physiological effects. For example, as demonstrated in the Examples below, a cocktail of four strains of wolf and dog probiotic bacteria displayed positive effects on intestinal barrier integrity and intestinal inflammation, while prebiotics such as maltodextrin showed greater effects on the intestinal microbiota composition and SCFA production than the strains themselves. Therefore, the probiotic and prebiotic components of the composition may have complementary effects to achieve an overall benefit in ameliorating symptoms of IBD and/or IBS.

The composition may be administered to the subject in an effective amount. As used herein, “effective amount” or “therapeutically effective amount” refers to an amount of the composition that can be effective in preventing, reducing or eliminating a symptom or health condition.

In some preferred embodiments, the composition is orally administrable to the subject. In other embodiments, the composition may be enterally and/or rectally administrable to the subject. In some embodiments, the composition may be administered to the subject at any suitable interval including, for example, at least once per month, at least once per week, or at least once per day.

In some embodiments, the effective amount may be administered as a single dose per day. In other embodiments, the effective amount may be administered in two or more sub-doses at appropriate intervals throughout the day, or as microdoses throughout the day. While it is preferred that the isolated strains and prebiotics be administered together as one dose, embodiments herein contemplate separate administration of one or more components of the composition.

In addition to its use in the compositions described herein, L. brevis WF-1B may be used alone as a probiotic to improve or maintain the health of a subject in a similar manner to the individual strains described in PCT/CA2019/051140. In some embodiments, L. brevis WF-1B may be used to treat or prevent intestinal dysbiosis in the subject or treat the subject for a health condition or disorder. In some embodiments, L. brevis WF-1B may be used to treat or prevent diarrhea in the subject. In other embodiments, L. brevis WF-1B may be used to provide any other health benefit to the subject. In some embodiments, L. brevis WF-1B may be used in the preparation of a medicament for treatment or prevention of intestinal dysbiosis, diarrhea, or any other suitable health condition.

In some embodiments, L. brevis WF-1B may be administered as part of a composition comprising the bacterial strain and one or more additional ingredients. The additional ingredients may include any of the ingredients described above for the multi-strain composition. Non-limiting examples of additional ingredients include one or more pharmaceutically or nutritionally acceptable excipients, encapsulation materials, edible ingredients and/or food products. The L. brevis WF-1B composition may be in any of the same forms as the composition described above, including, for example, supplements and food products.

Also provided herein is a method for preparing a composition for administration to a subject having IBD or IBS. The method may be used to prepare embodiments of the compositions disclosed herein.

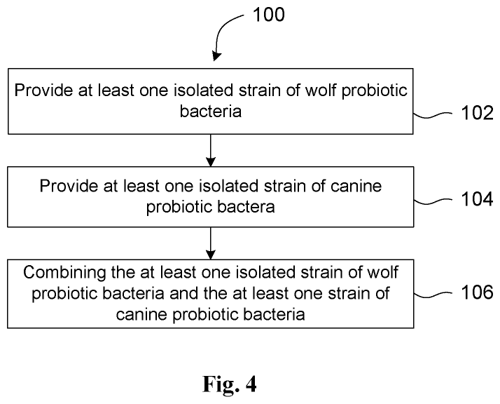

FIG. 4 shows a flowchart of an exemplary method 100 for making a composition, according to some embodiments. At block 102, at least one isolated strain of wolf probiotic bacteria is provided. At block 104, at least one isolated strain of canine probiotic bacteria is provided. The term “providing” in this context may refer to making (including isolating or culturing), receiving, buying, or otherwise obtaining the isolated strains.

The isolated strains of wolf probiotic bacteria and canine probiotic bacteria may be any of the strains disclosed herein. In some preferred embodiments, the isolated strains of wolf probiotic bacteria are L. brevis WF-1B IDAC Accession number 051120-02 and E. faecium strain WF-3 IDAC Accession number 181218-03; and the isolated strains of canine probiotic bacteria are L. casei strain K9-1 IDAC Accession number 210415-01 and L. fermentum strain K9-2 IDAC Accession number 210415-02.

At block 106, the isolated strain(s) of wolf probiotic bacteria are combined with the isolated strain(s) of canine probiotic bacteria. The term “combining” in this context refers to mixing, blending, or otherwise bringing together the isolated strains.

In some embodiments, the method 100 further comprises providing at least one prebiotic. For example, the prebiotic may comprise maltodextrin, humic acid and/or fulvic acid. In some embodiments, the method 100 further comprises combining the prebiotic(s) with the isolated strains of wolf and canine probiotic bacteria. In some embodiments, the isolated strains and prebiotic(s) are combined together at the same time. In other embodiments, the isolated strains are combined first to form a mixture and the mixture is combined with the prebiotic(s).

In some embodiments, the method 100 further comprises providing one or more additional ingredients and combining the additional ingredient(s) with the isolated strains and prebiotic(s). Non-limiting examples of additional ingredients include one or more pharmaceutically or nutritionally acceptable excipients, encapsulation materials, edible ingredients and/or food products.

Also provided herein is a kit comprising a composition in a container and instructions for administration of the composition to a subject having IBD and/or IBS. The composition may comprise at least one isolated strain of wolf probiotic bacteria and at least one isolated strain of canine probiotic bacteria. The isolated strains of wolf probiotic bacteria and canine probiotic bacteria may be any of the strains disclosed herein. In some preferred embodiments, the isolated strains of wolf probiotic bacteria are L. brevis WF-1B IDAC Accession number 051120-02 and E. faecium strain WF-3 IDAC Accession number 181218-03; and the isolated strains of canine probiotic bacteria are L. casei strain K9-1 IDAC Accession number 210415-01 and L. fermentum strain K9-2 IDAC Accession number 210415-02.

The isolated strains in the kit can be provided in a freeze-dried form, a liquid form, or in any other suitable form. Although the isolated strains are preferably combined in a single container, embodiments are also contemplated in which one or more strains are provided in separate containers and the kit includes instructions for combining the strains together.

In some embodiments, the composition further comprises at least one prebiotic including, for example, maltodextrin, humic acid, and/or fulvic acid. In some embodiments, prebiotic(s) are combined in the same container as the isolated strain. In other embodiments, at least one prebiotic may be provided in a separate container and the kit may include instructions for combining the prebiotic(s) with the rest of the composition.

The instructions for administration of the composition may comprise instructions for administering the composition to a companion animal such as a domestic dog. The instructions may include a recommended dosage and frequency for administering the composition and may also include instructions to take the composition with or without food, with or without other medications, etc.

Without any limitation to the foregoing, the present compositions, uses, and methods are further described by way of the following examples.

### Example 1—Isolation and Identification of L. brevis WF-1B

A feces sample from a free ranging wolf was collected from Prince Albert National Park in Saskatchewan, Canada on Mar. 23, 2017. A novel strain, labeled WF-1B, was isolated and identified using the methods described in PCT/CA2019/051140.

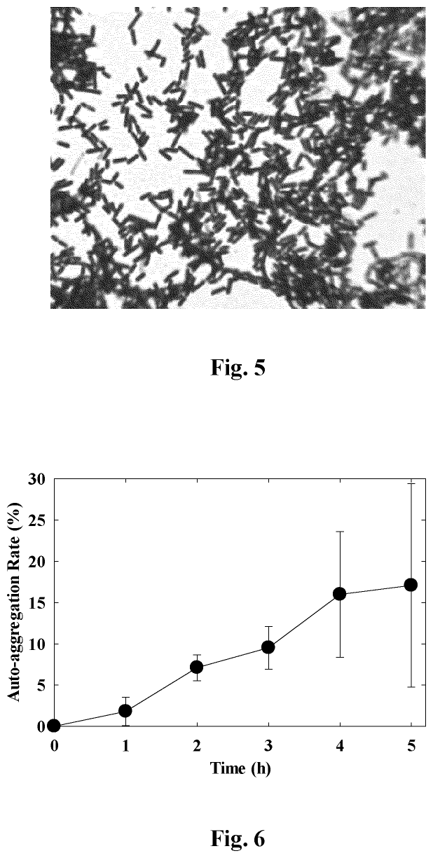

Gram staining was performed using standard methods and the gram-stained bacteria were visualized using a 100× lens on an OMAX™ LED 40×-2000× Digital Binocular Biological Compound Microscope and photos were obtained using a 3.0 MP USB camera connected to the microscope. The Gram staining results showing the rod-shaped bacterial morphology of isolated strain WF-1B are shown in FIG. 5.

To identify the species of the strain, the partial gene encoding the 16S ribosomal DNA (rDNA) was amplified by PCR and sequenced by Sanger Sequencing as described in described in PCT/CA2019/051140. The 16S rDNA sequencing results are shown in FIG. 2 and the isolated strain was identified as Levilactobacillus brevis.

To identify the isolate at the strain level, whole genome sequencing (Illumina™ Sequencing) was performed to get more detailed information about the strain. The data analysis results of the whole genome sequencing of L. brevis WF-1B are shown in Table 3 below.

TABLE 3

| | L. brevis WF-1B |
|---|---|
| Median genome size at species level | 2,570,500 |
| (bp) | |
| Sequencing strategy and | Illumina Novaseq 6000 |
| instrumentation used | (150 bp, paired end) |
| Software used for reads quality check | FastQC ™ |
| | (version 0.11.7) |
| Base calling Q score before trimming | 36 |
| (accuracy) | (99.97%) |
| # of reads in total before trimming | 7,160,318 |
| | (3,580,159 per end) |
| Average sequence length before | 150 |
| trimming (bp) | |
| Total base pairs of sequence data | 1,074,047,700 |
| before trimming (bp) | (537,023,850 per end) |
| Coverage depth of the genome | 417 |
| Software used for sequence trimming | Trimmomatic ™ |
| and adaptor removal | (version 0.36) |
| Parameters applied for sequence | ILLUMINACLIP:TruSeq3-PE- |
| trimming and adaptor removal | NovoG.fa:2:30:10 |
| | LEADING:20 TRAILING:20 |
| | SLIDINGWINDOW:4:20 |
| | AVGQUAL:20 MINLEN:75 |
| # of reads in total after trimming | 6,892,250 |
| | (3,446,125 per end) |
| Average sequence length after | 150 |
| trimming (bp) | |
| Total base pairs of sequence data | 1,033,837,500 |
| after trimming (bp) | |
| Software used for sequence assembling | SPAdes ™ |
| | (version 3.11.1) |
| Parameters applied for sequence | −k 21, 33, 55, 77, 99 |
| assembling | --cov-cutoff auto |
| Total # of contigs | 43 |
| # of contigs over 500 bp | 29 |
| Largest contig (bp) | 504,198 |
| Total length (bp) of contigs over | 2,683,271 |
| 500 bp | |
| Variation compared with the expected | 4% |
| genome size | |
| N50 metric | 394,318 |
| GC content | 45% |
| Software used for sequence annotation | RAST |
| | (version 2.0) |
| Parameters applied for sequence | Annotation scheme: RASTtk |
| annotation | Preserve gene calls: no |
| | Automatically fix errors: yes |
| | Fix frameshifts: yes |
| | Backfill gaps: yes |

Samples of a biologically pure culture of isolated strain L. brevis WF-1B were deposited in the International Depositary Authority of Canada (IDAC) (1015 Arlington Street, Winnipeg, Manitoba, Canada R3E 3R2) under the Budapest Treaty on Nov. 5, 2020 and assigned accession number 051120-02.

### Example 2—Characterization of L. brevis WF-1B

The biological activity of L. brevis WF-1B was characterized using the methods described in PCT/CA2019/051140 as outlined below.

### Example 2.1—Auto-Aggregation Ability

To assess the auto-aggregation activity of the isolate, auto-aggregation assays were performed. Thirty mL of fully-grown culture was mixed thoroughly by vortexing. The initial optical density at 600 nm (OD₆₀₀, A₀) was measured and recorded. The remaining cell suspension was kept still and undisturbed at ambient temperature for 5 hours. One hundred μL of the upper suspension (the cell suspension was not vortexed) was taken at one-hour intervals to measure the OD_{600 nm} (Aₜ). The auto-aggregation percentage was expressed as:

$$1 - \frac{{A}_{t}}{{A}_{o}}$$

wherein A₀ stands for OD₆₀₀ at 0 h, and Aₜ stands for OD₆₀₀ at 1 h, 2 h, 3 h, 4 h, or 5 h.

The auto-aggregation rate (in percentage) of L. brevis WF-1B is shown in FIG. 6. These results indicate that the isolate has the potential to adhere to host intestinal epithelial cell surface.

### Example 2.2—Cell Surface Hydrophobicity

To assess the hydrophobic nature of the bacterial cell surface of L. brevis WF-1B, microbial adhesion to hydrocarbons (MATH) assays (Otero et al., 2004) were performed to measure the hydrophobicity of the strain in terms of adhesion. Ten mL of fully-grown culture was harvested by centrifugation at 8,000 rpm for two minutes, followed by washing the cells with saline solution three times. The cell pellet was resuspended with saline solution and the OD₆₀₀ of each cell suspension was adjusted to 0.5±0.1. The actual final OD₆₀₀ of each cell suspension was measured and recorded. Three point six mL of cell suspension was aliquoted to a glass testing tube, followed by aliquoting 0.6 mL of solvent (toluene or xylene) to the same glass testing tube and vortexing vigorously for 1 minute. The testing tube was kept still for 1 hour to allow the immiscible solvent and aqueous phase to separate. The aqueous layer was removed with a Pasteur pipet and the OD₆₀₀ (ODₜₑₛₜ) was measured and recorded. The percentage of hydrophobicity of each strain was calculated as the following formula:

% hydrophobicity=(ODᵢₙᵢₜᵢₐₗ−ODₜₑₛₜ)/ODᵢₙᵢₜᵢₐₗ

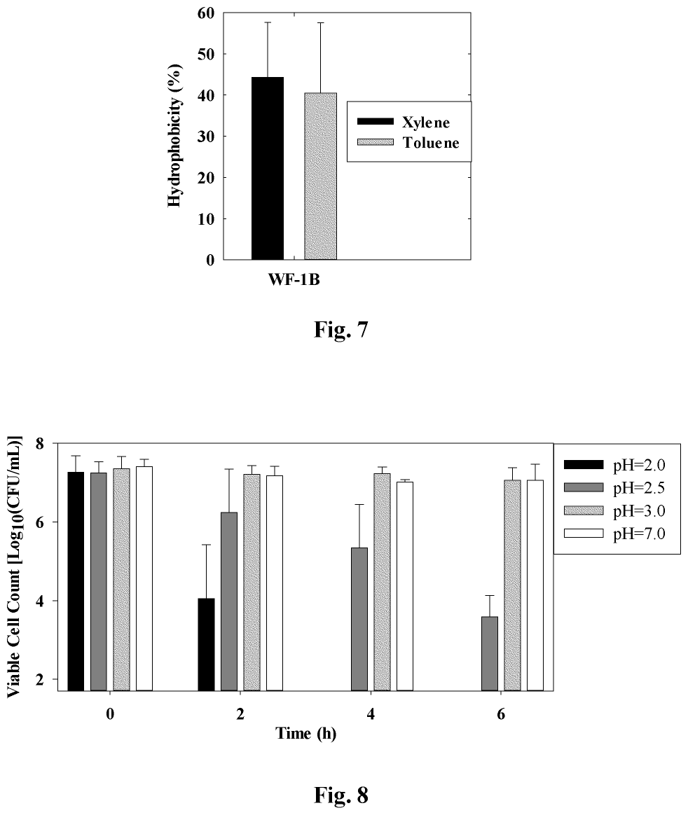

The percentage hydrophobicity of L. brevis WF-1B is shown in FIG. 7. These results indicate that the isolate has the potential to adhere to host intestinal epithelial cell surface.

### Example 2.3—Low pH and Bile Salt Tolerance Assays

To assess the tolerance of L. brevis WF-1B to acidic conditions, 1% of fully-grown culture (10 μL) was subcultured into a set of 1 mL solutions of Simulated Gastric Fluid (SGF, without pepsin) with varying pH values (pH=2.0, 2.5, 3.0, and 7.0). The SGF solutions with different pH values were prepared by adjusting the pH of SGF with HCl and NaOH, followed by sterilization by filtering. Once each subculture was inoculated into each SGF solution, the mixture was mixed thoroughly by vortexing and 60 μL of each mixture was aliquoted into the first column of a 96-well microtiter plate right away for diluting and plating. The remaining cultures were immediately incubated at 37° C. under airtight conditions for 6 h. Sixty μL of each culture was aliquoted into the first column of a new 96-well microtiter plate after 2 h, 4 h, and 6 h of incubation, respectively, for diluting and plating.

To assess the tolerance of the isolated strain to bile salt, 1% fully-grown culture (10 μL) was subcultured into a set of 1 mL of Phosphate Buffered Saline (PBS, pH=7.2) with varying bile salt concentrations (0%, 3%, and 5%). The PBS solutions with different bile salt concentrations were prepared by dissolving a corresponding amount of bile salt into sterile PBS. Once a culture was inoculated into each PBS solution, the mixture was mixed thoroughly by vortexing and 60 μL of each mixture was aliquoted into the first column of a 96-well microtiter plate right away for diluting and plating. The remaining cultures were immediately incubated at 37° C. under airtight conditions for 24 h. Sixty μL of each culture was aliquoted into the first column of a new 96-well microtiter plate after 6 h and 24 h of incubation, respectively, for diluting and plating.

A serial 10-fold dilution of each culture was prepared and proper dilutions were plated on MRS agar plates and incubated at 37° C. for 2 days. Viable cell counts were recorded and expressed as the Mean [log₁₀(CFU/mL)]±Standard Error of at least three independent replicates.

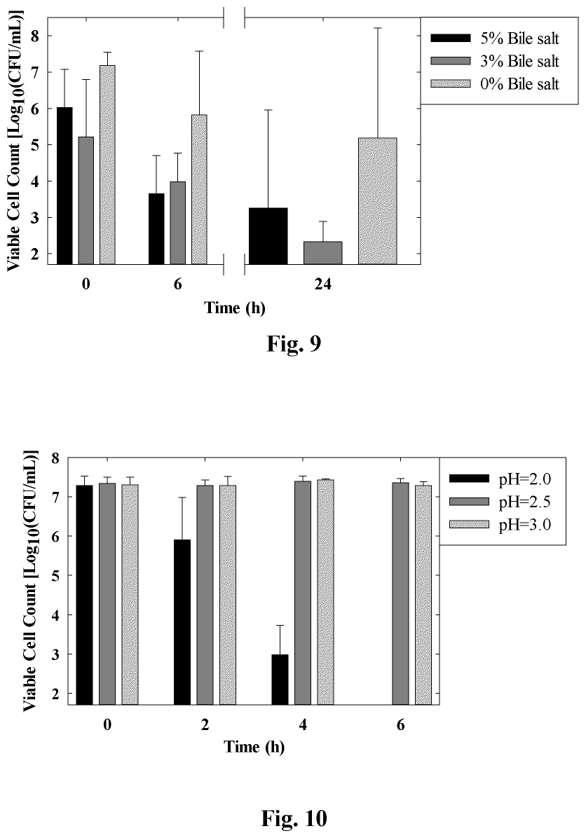

The results of the low pH and bile salt tolerance assays for L. brevis WF-1B are shown in FIGS. 8 and 9, respectively. The low pH study showed that WF-1B survived in a solution at pH 2 for 2 hours and survived in solutions at pH 2.5 and 3.0 for 6 hours. The bile salt tolerance assay showed that WF-1B survived at 3% and 5% bile salt for 24 hours.

### Example 2.4—Gastric and Intestinal Digestive Enzyme Tolerance Assays

To assess the tolerance of L. brevis WF-1B to gastric digestive enzyme, 1% fully-grown culture (10 μL) was subcultured into a set of 1 mL of SGF solutions (with 3.2 mg/mL of pepsin) with varying pH values (pH=2.0, 2.5, and 3.0). The cultures were incubated at 37° C. under airtight conditions for 6 h. Sixty μL of each culture was aliquoted into the first column of a 96-well microtiter plate after 0 h, 2 h, 4 h, and 6 h of incubation, respectively, for diluting and plating.

To assess the tolerance of the isolate to intestinal digestive enzyme, 1 fully-grown culture (10 μL) was subcultured into a set of 1 mL of Simulated Intestinal Fluid (SIF) solutions with 10 mg/mL of pancreatin at pH=6.8. The cultures were incubated at 37° C. under airtight conditions for 24 h. Sixty μL of each culture was aliquoted into the first column of a 96-well microtiter plate after 0 h, 6 h, and 24 h of incubation, respectively, for diluting and plating.

A serial 10-fold dilution of each culture was prepared and proper dilutions were plated on MRS agar plates and incubated at 37° C. for 2 days. Viable cell counts were recorded and expressed as the Mean [log₁₀(CFU/mL)]±Standard Error of at least three independent replicates.

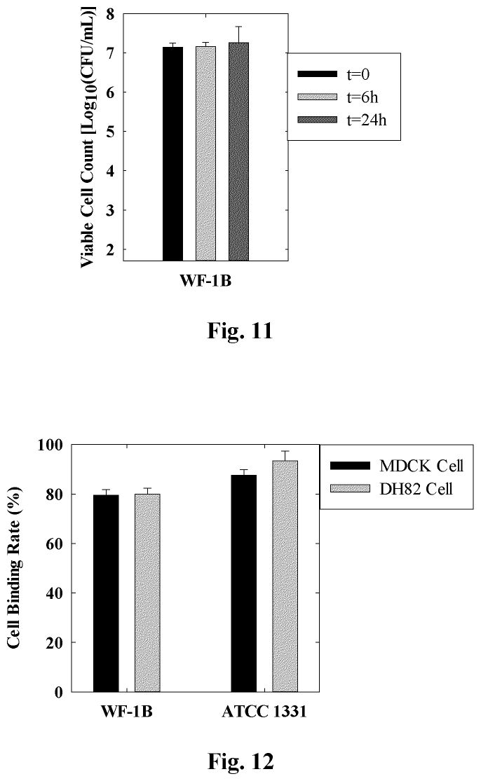

The results of the gastric digestive enzyme and intestinal digestive enzyme tolerance assays for L. brevis WF-1B are shown in FIGS. 10 and 11, respectively. The gastric digestive enzyme tolerance assay showed that WF-1B survived in SGF (with 3.2 mg/mL of pepsin) at pH 2.0 for 4 h and at pH 2.5 and 3.0 for 6 hours. The intestinal digestive enzyme tolerance assay showed that WF-1B survived in a SIF (with 10 mg/mL of pancreatin) for 24 h.

### Example 2.5—Production of Inhibitory Substances

To assess the ability of L. brevis WF-1B to produce any inhibitory substances against a series of pathogenic and spoilage microorganisms, the isolate was grown in the presence of a series of indicator strains. One μL of fully-grown culture was spotted on Reinforced Clostridial Agar (RCA) plates and incubated at 37° C. overnight. Ten indicator strains were cultivated in Trypticase Soy Broth with 0.6% Yeast Extract (TSBYE) at 37° C. overnight. Each indicator strain (0.1%, 6 μL) was inoculated into 6 mL of RCA soft agar (with 0.75% agar), followed by pouring the mixture on top of the spotted RCA plates. The solidified agar plates were incubated at 37° C. overnight. The inhibitory zone size without visible growth of indicator strains was measured and recorded.

The results are shown in Table 4. In Table 4: “Yes” indicates that an isolate produces inhibitory substances against the corresponding indicator strain; “No” indicates that the strain does not produce inhibitory substances against the corresponding indicator strain; “MRSA” refers to methicillin resistant Staphylococcus aureus; and “VRE” refers to vancomycin-resistant Enterococcus.

TABLE 4

| Indicator strains | L. brevis WF-1B |
|---|---|
| E. coli ATCC 11775 | Yes |
| E. coli ATCC 25927 | Yes |
| S. enterica ATCC 13311 | Yes |
| S. enterica ATCC 8326 | Yes |
| L. monocytogenes ATCC 1946 | Yes |
| L. monocytogenes ATCC 43256 | Yes |
| MRSA R667 | Yes |
| MRSA R776 | Yes |
| VRE R704 | Yes |
| VRE R846 | Yes |

As shown in Table 4, WF-1B produced inhibitory substances against all 10 indicator strains tested in this study.

### Example 2.6—Antibiotic Susceptibility Assay and Sequence Analysis

Broth microdilution was used to determine the susceptibility of the L. brevis WF-1B isolate against eight commonly used clinical antibiotics. Broth micro-dilution was performed following the methods according to: International Organization for Standardization, Milk and milk products—Determination of the minimal inhibitory concentration (MIC) of antibiotics applicable to bifidobacteria and non-enterrococcal lactic acid bacteria (LAB) (ISO 10932:2012). Antibiotic stock solutions were prepared following the methods according to: CLSI, Performance Standards for Antimicrobial Susceptibility Testing, 23^{rd} edition, CLSI Standard M100, Wayne, Pa.: Clinical and Laboratory Standards Institute; 2013.

The measured minimum inhibitory concentrations (MICs) and microbiological cut-off values from the antibiotic susceptibility assays for L. brevis WF-1B are shown below in Table 5.

TABLE 5

| | |
|---|---|
| Antibiotics | L. brevis WF-1B |

| (μg/mL) | MIC | Cut-off value |
|---|---|---|
| Ampicillin | 8 | 2 |
| Gentamicin | 3 | 16 |
| Kanamycin | 85 | 32 |
| Streptomycin | 8 | 64 |
| Erythromycin | 0.83 | 1 |
| Clindamycin | 8 | 1 |
| Tetracycline | 64 | 8 |
| Chloramphenicol | 16 | 4 |

As shown in Table 5, WF-1B is susceptible to several antibiotics including gentamicin, streptomycin, and erythromycin, for which the MICs are below the European Food Safety Authority (EFSA) cut-off values.

To investigate the nature of resistance, firstly the MIC distribution was summarized at species level. Secondly, the whole genome shotgun sequence (contigs or scaffolds) was interrogated for the presence of genes coding for or contributing to resistance to any antimicrobials that are of clinic importance by comparing against a list of up-to-date databases, including comprehensive antibiotic resistance database (CARD), antibiotic resistance gene annotation database (ARG-ANNOT), ReFinder 4.1, and Rapid Annotation Using Subsystem Technology (RAST).

L. brevis WF-1B was sensitive to gentamicin, streptomycin, and erythromycin, but resistant to ampicillin, kanamycin, clindamycin, tetracycline, and chloramphenicol. The MICs of ampicillin, kanamycin, clindamycin, and chloramphenicol against L. brevis WF-1B fell in the MIC distribution ranges at the species level for L. brevis, which indicates these resistances likely belong to intrinsic or natural resistance. The MIC of tetracycline against L. brevis WF-1B fell out of the MIC distribution ranges at the species level for L. brevis, which indicates the tetracycline resistance of L. brevis WF-1B belongs to acquired resistance.

No hits were found for L. brevis WF-1B by comparing with databases CARD by performing RGI (resistance genes identifier) analysis, ResFinder 4.1 by searching acquired antimicrobial resistance genes, and ARG-ANNOT by performing blast.

Moreover, virulence factors, antibiotic resistance, and transposable elements were annotated by searching the Subsystem Feature Counts of the RAST output for those factors identified in the Virulence, Disease and Defense subsystem, and Prophages, Transposable Elements, and Plasm ids subsystem. No virulence factors or pathogenicity islands were identified in L. brevis WF-1B. The antibiotic resistance (AR) determinants identified in L. brevis WF-1B include translation elongation factor G, ribosome protection-type tetracycline resistance related proteins (group 2), DNA gyrase subunit A and B, transcription regulator of multidrug efflux pump operon, TetR (AcrR) family, multi antimicrobial extrusion protein (Na(+)/drug antiporter), and MATE family of MDR efflux pumps.

Thus, L. brevis WF-1B was resistant to the antibiotics listed above due to the presence of ribosome protection-type tetracycline resistance related proteins (group 2), translation elongation factor G, and multidrug resistance efflux pumps, which were present on the chromosomes of L. brevis WF-1B instead of presence on the plasmids. Moreover, the upstream and downstream sequences flanking the genes listed above were characterized by comparing them with that of similar organisms and no mobile genetic elements were identified. Additionally, no transposable elements and gene transfer agents were identified in L. brevis WF-1B. Therefore, the resistance is classified as either intrinsic resistance or acquired resistance due to genomic mutation. The risk of horizontal AR gene transfer is low. Therefore, it is considered safe to use L. brevis WF-1B as a feed additive in animal nutrition.

### Example 2.7—Cell Binding Assay

To assess the adhesion ability of the L. brevis WF-1B isolate in vitro, two canine cell lines, MDCK and DH82, were used in this study. Canis familiaris ATCC CCL-34 (MDCK (NBL-2)) and Canis familiaris ATCC CRL-10389 (DH82) were resuscitated from frozen stocks stored in a liquid nitrogen tank with a complete medium in a tissue culture flask. The base medium used in this study for cell line cultivation was DMEM (Dubecco's Modified Eagle Media; Gibco™) with high glucose level, glutamine, and sodium pyruvate. The complete medium was composed of DMEM and 10% heat-inactivated (56° C. for 30 min) fetal bovine serum (FBS; Gibco™). The growth condition was 37° C. with 5% CO₂. The solution used for cell dispersion was 0.25% (w/v) Trypsin with 0.53 mM EDTA (ethylenediaminetetraacetic acid). The cell line cultures were maintained for two weeks after the confluence to allow full differentiation before the adhesion assay. A hemocytometer was used for cell counting.

Bacterial cell suspensions were prepared by harvesting 5 mL of fully-grown culture by centrifugation at 3,500 g for 10 min, followed by washing cells with PBS (pH=7.4) three times. The cell pellet was resuspended in base medium DMEM and adjusted to an OD₆₀₀ nm of around 1.0 for the WF-1B isolate and around 0.1 for control strain S. enterica ATCC 13311 which corresponds to about 5×10⁸ CFU/mL for the WF-1B isolate and about 1×10⁸ CFU/mL for the control strains.

Cell monolayers of MDCK and DH82 cells were prepared in 12-well tissue culture plates. Cells were inoculated at a concentration of 4×10⁴ cells per well to obtain confluence and allowed to differentiate. The culture medium was changed every two days. Once the cells were confluent, the complete medium was removed followed by washing cells with PBS for three times. One mL of base medium DMEM was added to each well and incubated at 37° C. with 5% CO₂ for 1 h before the adhesion assay.

A 1 mL aliquot of bacterial cell suspension was added to the confluent monolayer cells and incubated at 37° C. with a 5% CO₂ atmosphere for 2 h. One mL of base medium DMEM was added to one well to serve as a sterility control. Two hours later, the monolayer cells were washed with PBS for three times. Two hundred fifty μL of Trypsin-EDTA solution was added to each well until cell layer was dispersed, followed by adding 1.75 mL of complete medium and aspirating cells by pipetting.

A serial 10-fold dilution of each culture was prepared and proper dilutions were plated on MRS agar plates and incubated at 37° C. for 2 days. Viable cell counts were recorded and expressed as the Mean [log₁₀(CFU/mL)]±Standard Error of at least three independent replicates. The cell binding rate was calculated as the viable cell count that bound to cell lines over the original inoculated CFU of the bacterial cell suspensions to the cell line.

The results of the cell binding assays are shown in FIG. 12. The cell binding assay results demonstrated that L. brevis WF-1B shows high cell surface binding capability.

### Example 3—Biological Effects of Wolf and Canine Isolated Strains and Prebiotics

### Example 3.1—Dog Feeding Trials

Three independent dog feeding trials, one conducted in Canada and two conducted in The Republic of Ireland, demonstrated that a composition containing four strains of lactic acid bacteria, L. casei K9-1, L. fermentum K9-2, L. brevis WF-1B, and E. faecium WF-3, was well tolerated in healthy Beagle dogs when administered orally once daily for 28 days.

Additionally, viable cell enumeration from faecal samples collected from a dog feeding trial by PMA-qPCR (Propidium monoazide—quantitative polymerase chain reaction) technology demonstrated that all four probiotic strains (L. casei K9-1, L. fermentum K9-2, L. brevis WF-1B, and E. faecium WF-3) successfully survive passage through the dog gastrointestinal tract.

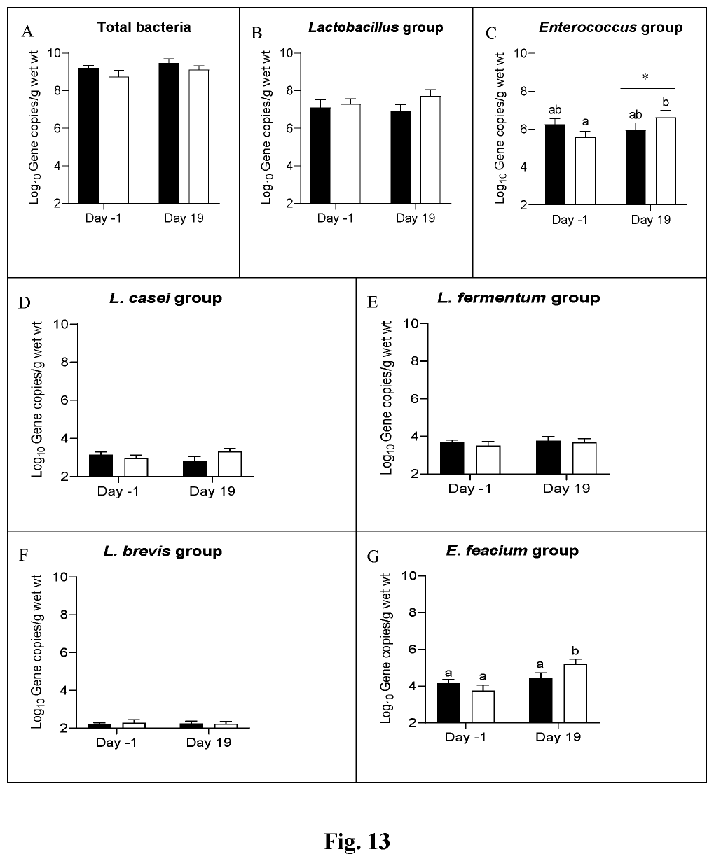

The effect of the composition on the abundance of specific bacterial species, including L. casei, L. fermentum, L. brevis, and E. faecium, in healthy dogs was determined by qPCR and the results are shown in FIG. 13. In FIG. 13, vertical bars represent means±SEM and data analyses show that no statistically significant difference was observed either between control (dogs fed with a placebo) and test groups (dogs fed with K-9 Heritage Probiotic Blend®) or between Day −1 (before treatment) and D19 (treatment Day 19) samples collected from the same testing group for total number of bacteria, Lactobacillus spp, L. casei, L. fermentum and L. brevis. The number of Enterococcus spp. present in faecal samples collected on D19 from the test group was significantly higher than that collected on Day −1 from test group (P<0.05) and that collected on D19 from control group (P<0.10). The number of E. faecium present in faecal samples collected on D19 from the test group was significantly higher than that collected on Day −1 from both control and test groups (P<0.05) and that collected on D19 from control group (P<0.05).

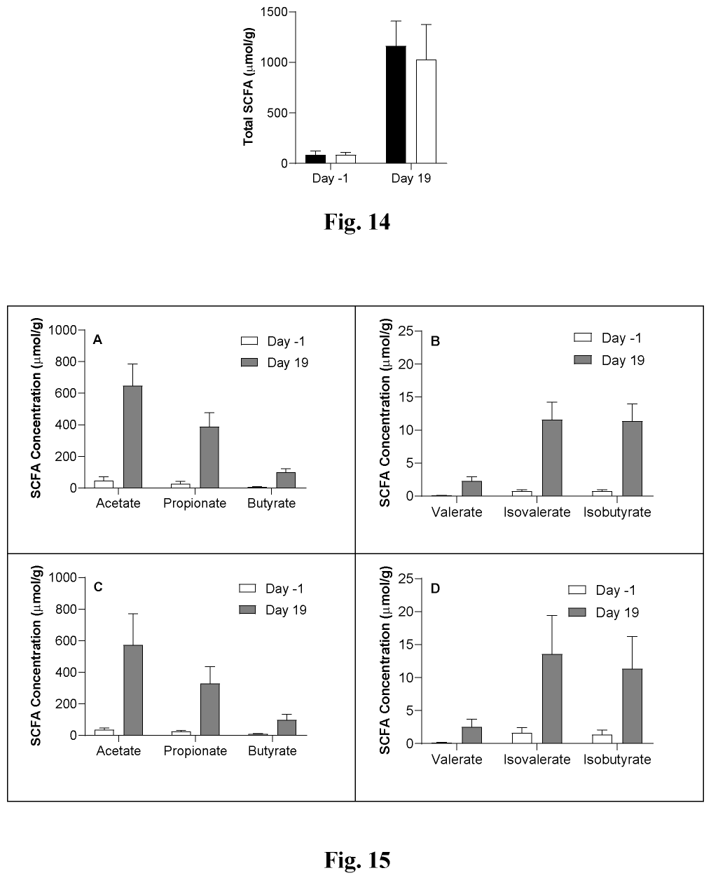

The effect of the composition on the production of short-chain fatty acids (SCFAs), including acetic acid, propionic acid, n-butyric acid, iso-butyric acid, valeric acid and iso-valeric acid, in healthy dogs was determined as well. The results are shown in FIGS. 14 and 15. Data analysis showed that the total quantity of SCFAs, including acetic acid, propionic acid, n-butyric acid, iso-butyric acid, valeric acid and iso-valeric acid, present in faecal samples collected on Day −1 from control and test groups was about 200 μmol/g of faeces. The total quantity of SCFAs present in faecal samples collected on Day 19 from control and test groups increased significantly to about 1,200 μmol/g of faeces and about 1,000 μmol/g of faeces, respectively. Overall, no significant difference was observed in terms of both total quantity of SCFAs or individual SCFA present in faecal samples collected on either Day −1 or Day 19 between control and test groups. However, the quantity of total SCFAs or individual SCFA (except for valeric acid) present in faecal samples collected from either control or test group increased dramatically from Day −1 to Day 19.

### Example 3.2—In Vitro Gastrointestinal Model

The survival of L. casei K9-1, L. fermentum K9-2, L. brevis WF-1B, and E. faecium WF-3 during passage through the canine stomach and small intestine was simulated in a dynamic in vitro gastrointestinal model simulating canine conditions referred to as TIM-1. The TIM-1 system was developed by TNO (The Netherlands Organization for Applied Scientific Research), The Netherlands, and is a computer-controlled model that simulates the physiological processes and conditions within the gastrointestinal tract. The TIM-1 system consists of several compartments interconnected by valves regulating GI transit.

The four strains, in lyophilized powder format mixed with a dry canine diet (kibble), were fed to the TIM-1 system, and viable cell equivalents were determined in the ileum effluent by PMA-qPCR technology. Results show that the survival rate of L. casei K9-1 after transit through TIM-1 was 95.6±4.0%, and 2.9±1.4% for L. fermentum K9-2, and 317±15% for L. brevis WF-1B, and 255±120% for E. faecium WF-3. These data demonstrate that the strains are capable of surviving passage through the canine GI tract and reaching the large intestines.

### Example 3.3—In Vitro Intestinal Tissue Model

The effect of L. casei K9-1, L. fermentum K9-2, L. brevis WF-1B, and E. faecium WF-3 on gut epithelial barrier functions and anti-inflammatory response of dog intestinal tissue was studied in an in vitro intestinal model (InTESTine™ platform, TNO, The Netherlands) with a segment of colon tissue from a healthy dog mounted in the platform. A Salmonella enterica strain was used as a pro-inflammatory agent and Cytochalasin D was used as a gut barrier function disturber.

The inoculation of S. enterica significantly disrupted the barrier function of colon tissue with specific effects on tight junction functioning. The increased paracellular transport of mannitol (a paracellular transport indicator) was decreased 10-15% when a cocktail of the four probiotic strains, L. casei K9-1, L. fermentum K9-2, L. brevis WF-1B, and E. faecium WF-3, was inoculated 30 min prior to the inoculation of S. enterica, indicating that these probiotic strains have positive effects on gut tight junction protein function and restoring or preventing barrier disturbances of the intestinal tissue.

The cumulative lactate dehydrogenase (LDH, a cell toxicity indicator) leakage into the apical and basolateral compartment was low for all of the incubations indicating proper intestinal tissue viability during the 6 hours of incubation. All incubations with the addition of a cocktail of four probiotic strains, L. casei K9-1, L. fermentum K9-2, L. brevis WF-1B, and E. faecium WF-3, showed reduced LDH release, indicating that these probiotic strains have a positive effect on maintaining the intestinal tissue viability. This positive effect was mainly caused by a 3- to 4-fold reduction of LDH secretion into the apical compartment.

The gene expression of IL-4, IL-6, IL-12a, IL-128, IFN-γ, and TNF-α and GAPDH in colon tissues was determined by qPCR. A trend of increased expression of IL-6, IL-12 β, IFN-γ, and TNF-α in the incubations with Salmonella enterica was observed. Interestingly, the increased expression of these cytokine genes was slightly diminished when a cocktail of four probiotic strains, L. casei K9-1, L. fermentum K9-2, L. brevis WF-1B, and E. faecium WF-3, was inoculated 30 min prior to the inoculation of S. enterica. In particular, the expression of TNF-α was significantly reduced. These results suggest that the four probiotic strain mix has a positive effect on the reduction of inflammatory reactions in the intestine induced by Salmonella enterica.

### Example 3.4—In Vitro Intestinal Microbiota Model

The effect of the probiotic strains and prebiotics on the production of short-chain-fatty acid (SCFA) and the shift of microbiota composition in canine colon was determined in an in vitro intestinal model (i-Screen™ platform, TNO, The Netherlands). Faecal materials donated by six healthy dogs were used for the preparation of basic inoculum for i-screen. One single probiotic strain or a cocktail of multiple probiotic strains with or without the addition of a mix of humic acid and fulvic acid or maltodextrin were inoculated into one well out of 96 wells of the i-screen incubation system. The production of SCFA was quantified by Gas Chromatography (GC) and the composition of faecal microbiota was determined by 16S rDNA gene amplicon sequencing of the V4 hypervariable region after 24 hours of incubation.

Data analyses showed that maltodextrin and to a lesser extent the presence of humic and fulvic acids supported the production of propionate at the expense of acetate. Maltodextrin also yielded high production of butyrate. The probiotic strains with the exception of E. faecium WF-3 gave rise to lesser changes to the SCFA production and the levels are more comparable to the control conditions (microbiota only). However, the presence of E. faecium WF-3 alone or in combination with L. brevis WF-1B or Lactilactobacillus curvatus WF-6 at an initial count of 10⁷ CFU/mL supported higher production of acetate compared to the other exposure conditions.

After 24 hours of incubation at 38° C., the relative abundance of the lactobacilli and enterococci in the microbiota changed. Specifically, the lactobacilli strains appeared not to colonize the canine gut microbiota in the i-screen at a high relative abundance, but rather they remained at a marginal percentage in the microbiota after 24 hours of incubation. On the other hand, Enterococcus faecium remained present in the canine gut microbiota in the i-screen at a higher level compared to lactobacilli. The prebiotic maltodextrin strongly affected the microbiota composition, while the mixture of humic and fulvic acids did so to a much lesser extent. Maltodextrin, particularly at the concentration of 10 mg/mL, supported the increase of genus Prevotella, Meganomonas, Phascolarctobaterium, Succinivibrio and Clostridium sensu stricto. This took place at the expense of Clostridium XI, Fusobacterium, Bacteroides, Parasutterella, Lachnospiraceae unclassified, and Dorea.

### Example 4—Summary of Previous Animal Feeding Trials with Humic acid and/or Fulvic Acid

Animal feeding trials with humic acid and/or fulvic acid conducted by other researchers demonstrated that humic acid and fulvic acid provide a number of different beneficial effects, including: maintaining or modulating gut microbiota; suppressing the growth of undesirable gut microbes but stimulating the growth of desirable gut microbes; reducing mold growth and toxin production; augmenting immune potency; improving gut health; improving nutrient digestibility and utilization; acting as a growth promoter; improving productive performance; reducing blood lipids and cholesterol; and increasing antioxidant capacity. (Islam et al., 2005; Kühnert et al., 2015; van Rensburg, 2015; Kaevska et al., 2016; Arif et al., 2019; Visscher et al., 2019; Mudron̆ová et al., 2020).

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

The following references are hereby incorporated by reference in their entirety:
Arif, M., Alagawany, M., El-Hack, M. A., Saeed, M., Arain, M. A., & Elnesr, S. S. (2019). Humic acid as a feed additive in poultry diets: a review. Iranian Journal of Veterinary Research, 20(3), 167.
Blain, A. H., Carlson, D. R., Miyata-Kane, S. T., & Stiles, M. E. (2015). Probiotic strains isolated from dogs for use in dog food, treats and/or supplements. Canadian Patent No. CA2890965C. Edmonton, Canada. Canadian Intellectual Property Office.
Islam, K. M. S., Schuhmacher, A., & Gropp, J. M. (2005). Humic acid substances in animal agriculture. Pakistan Journal of nutrition, 4(3), 126-134.
Kaevska, M., Lorencova, A., Videnska, P., Sedlar, K., Provaznik, I., & Trckova, M. (2016). Effect of sodium humate and zinc oxide used in prophylaxis of post-weaning diarrhoea on faecal microbiota composition in weaned piglets. Veterinárni Medicina, 61(6), 328-336.
Kühnert, M., Kruger, M., Haufe, S., & Sheata, A. (2015). Use of a humic acid preparation for treating warm-blooded animals. International Patent Application No. WO2014040590A1.
Major, G., & Spiller, R. (2014). Irritable bowel syndrome, inflammatory bowel disease and the microbiome. Current Opinion in Endocrinology, Diabetes, and Obesity, 2/(1), 15.
Mudron̆ová, D., Karaffová, V., Pes̆ulová, T., Kos̆c̆ová, J., Marus̆c̆áková, I. C., Bartkovský, M., Marcinc̆áková, D., S̆evc̆iková, Z., & Marcinc̆ák, S. (2020). The effect of humic substances on gut microbiota and immune response of broilers. Food and Agricultural Immunology, 31(1), 137-149.
Otero et al. (2004) “Bacterial surface characteristics applied to selection of probiotic microorganisms”, in Public Health Microbiology, pp. 435-440. Humana Press.
van Rensburg, C. E. (2015). The antiinflammatory properties of humic substances: a mini review. Phytotherapy Research, 29(6), 791-795.
Visscher, C., Hankel, J., Nies, A., Keller, B., Galvez, E., Strowig, T., Keller, C., & Breves, G. (2019). Performance, fermentation characteristics and composition of the microbiome in the digest of piglets kept on a feed with humic acid-rich peat. Frontiers in Veterinary Science, 6, 29.

1. A composition comprising:
a first isolated strain of wolf probiotic bacteria, wherein the first isolated strain of wolf probiotic bacteria is a species of the Lactobacillaceae family;
a second isolated strain of wolf probiotic bacteria, wherein the second isolated strain of wolf probiotic bacteria is a species of the Enterococcaceae family; and
at least one isolated strain of canine probiotic bacteria, wherein the at least one isolated strain of canine probiotic bacteria comprises at least one species of the Lactobacillaceae family.

2. The composition of claim 1, further comprising at least one prebiotic.

3. The composition of claim 1 wherein the at least one prebiotic comprises at least one of maltodextrin, humic acid, and fulvic acid.

4. The composition of claim 1, wherein the first isolated strain of wolf probiotic bacteria is a Levilactobacillus species and the second isolated strain of wolf probiotic bacteria is an Enterococcus species.

5. The composition of claim 4, wherein the first isolated strain of wolf probiotic bacteria is Levilactobacillus brevis and the second isolated strain of wolf probiotic bacteria is Enterococcus faecium.

6. The composition of claim 5, wherein the first isolated strain of wolf probiotic bacteria is Levilactobacillus brevis WF-1B IDAC Accession number 051120-02 or a mutant strain thereof and wherein the second isolated strain of wolf probiotic bacteria is Enterococcus faecium strain WF-3 IDAC Accession number 181218-03 or a mutant strain thereof.

7. The composition of claim 1, wherein the at least one isolated strain of canine probiotic bacteria comprises a Lacticaseibacillus species and a Limosilactobacillus species.

8. The composition of claim 7, wherein the at least one strain of canine probiotic bacteria comprises Lacticaseibacillus casei and Limosilactobacillus fermentum.

9. The composition of claim 8, wherein the at least one isolated strain of canine probiotic bacteria comprises: Lacticaseibacillus casei strain K9-1 IDAC Accession number 210415-01 or a mutant strain thereof; and Limosilactobacillus fermentum strain K9-2 IDAC Accession number 210415-02 or a mutant strain thereof.

10. The composition of claim 1, wherein the composition comprises:
Levilactobacillus brevis strain WF-1B IDAC Accession number 051120-02;
Enterococcus faecium strain WF-3 IDAC Accession number 181218-03;
Lacticaseibacillus casei strain K9-1 IDAC Accession number 210415-01;
Limosilactobacillus fermentum strain K9-2 IDAC Accession number 210415-02;
at least one of maltodextrin, humic acid, and fulvic acid.

11-12. (canceled)

13. A method for treating IBD and/or IBS in a subject comprising administering a composition of to the subject, the composition comprising:
a first isolated strain of wolf probiotic bacteria, wherein the first isolated strain of wolf probiotic bacteria is a species of the Lactobacillaceae family;
a second isolated strain of wolf probiotic bacteria, wherein the second isolated strain of wolf probiotic bacteria is a species of the Enterococcaceae family; and
at least one isolated strain of canine probiotic bacteria, wherein the at least one isolated strain of canine probiotic bacteria comprises at least one species of the Lactobacillaceae family.

14. The method of claim 13, wherein the subject is a domestic dog.

15. The method of claim 13, wherein the composition is administered orally.

16. (canceled)

17. A method for making a composition for treating IBD and/or IBS, comprising:
providing a first isolated strain of wolf probiotic bacteria, wherein the first isolated strain of wolf probiotic bacteria is a species of the Lactobacillaceae family;
providing a second isolated strain of wolf probiotic bacteria, wherein the second isolated strain of wolf probiotic bacteria is a species of the Enterococcaceae family;
providing at least one isolated strain of canine probiotic bacteria, wherein the at least one isolated strain of canine probiotic bacteria comprises at least one species of the Lactobacillaceae family; and
combining the first and second isolated strains of wolf probiotic bacteria and the at least one strain of canine probiotic bacteria.

18. The method of claim 17, further comprising providing at least one prebiotic and combining the at least one prebiotic with the first and second isolated strains of wolf probiotic bacteria and the at least one isolated strain of canine probiotic bacteria.

19-22. (canceled)

23. The composition of claim 1, wherein the first isolated strain of wolf probiotic bacteria is Levilactobacillus brevis WF-1B IDAC Accession number 051120-02.

24. The method of claim 13, wherein the first isolated strain of wolf probiotic bacteria is Levilactobacillus brevis WF-1B IDAC Accession number 051120-02.

25. The method of claim 13, wherein the composition further comprises at least one prebiotic selected from maltodextrin, humic acid, and fulvic acid.

26. The method of claim 17, wherein the first isolated strain of wolf probiotic bacteria is Levilactobacillus brevis WF-1B IDAC Accession number 051120-02.

27. The method of claim 18, wherein the at least one prebiotic comprises at least one of maltodextrin, humic acid, and fulvic acid.


SEQUENCE LISTING (10 sequences)

SEQ ID NO: 1 - DNA, 767 bp, Lactobacillus reuteri
  misc_feature (456)..(456): n is a, t, g, or c
  misc_feature (561)..(561): r is a or g
agcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattccctactgctgcctcccgtaggagtatggaccgtgtctcagttccattgtggccgatcagnctctcaactcggctatgcatcatcgccttggtaagccgttaccttaccaactagctaatgcaccgcaggtccatcccagagtgatagccaaagccatctttcaarcaaaagccatgtggcttttgttgttatgcggtattagcatctgtttccaaatgttatcccccgctccggggcaggttacctacgtgttactcacccgtccgccactcactggtgatccatcgtcaatcaggtgcaagcaccatcaatcagttgggccagtgcgtacgacttgcatgtattaggcacaccgccggcgttcatcctga

SEQ ID NO: 2 - DNA, 692 bp, Lactobacillus animalis
ttacagaccagagagccgctttcgccactggtgttcttccatatatctacgcatttcaccgctacacatggagttccactctcctcttctgcactcaagtctcccagtttccaatgcactactccggttaagccgaaggctttcacatcagacttaaaagaccgcctgcgttccctttacgcccaataaatccggataacgcttgccacctacgtattaccgcggctgctggcacgtagttagccgtggctttctggttagataccgtcgaaacgtgaacagttactctcacgcactttcttctctaacaacagggttttacgatccgaagaccttcttcacccacgcggcgttgctccatcaggctttcgcccattgtggaagattccctactgctgcctcccgtaggagtttgggccgtgtctcagtcccaatgtggccgatcaacctctcagttcggctacgcatcattgccttggtaagcctttacctcaccaactagctaatgcgccgcgggcccatccaaaagcggtagcatagccaccttttacatagttaccatgcggtaactatggttatgcggtattagcacctgtttccaagtgttatccccctcttttgggcaggttgcccacgtgttactcacccgttcgccactcaactctttatcggtgagtgcaagcactcggtga

SEQ ID NO: 3 - DNA, 706 bp, Enterococcus faecium
  misc_feature (558)..(558): n is a, t, g, or c
agccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaagggatgaacagttactctcatccttgttcttctctaacaacagagttttacgatccgaaaaccttcttcactcacgcggcgttgctcggtcagactttcgtccattgccgaagattccctactgctgcctcccgtaggagtttgggccgtgtctcagtcccaatgtggccgatcaccctctcaggtcggctatgcatcgtggccttggtgagccgttacctcaccaactagctaatgcaccgcgggtccatccatcagcgacacccgaaagcgcctttcaaatcaaaaccatgcggtttngattgttatacggtattagcacctgtttccaagtgttatccccttctgatgggcaggttacccacgtgttactcacccgttcgccactcctctttttccggtggagcaagctccggtggaaaaagaagcgttcgacttgcatgtatta

SEQ ID NO: 4 - DNA, 760 bp, Lactobacillus plantarum
  misc_feature (109)..(109): y is c or t
  misc_feature (146)..(146): n is a, t, g, or c
  misc_feature (561)..(561): n is a, t, g, or c
  misc_feature (734)..(734): n is a, t, g, or c
agcgtcagttacagaccagacagccgccttcgccactggtgttcttccatatatctacgcatttcaccgctacacatggagttccactgtcctcttctgcactcaagtytcccagtttccgatgcacttcttcggttgagccgaangctttcacatcagacttaaaaaaccgcctgcgctcgctttacgcccaataaatccggacaacgcttgccacctacgtattaccgcggctgctggcacgtagttagccgtggctttctggttaaataccgtcaatacctgaacagttactctcagatatgttcttctttaacaacagagttttacgagccgaaacccttcttcactcacgcggcgttgctccatcagactttcgtccattgtggaagattccctactgctgcctcccgtaggagtttgggccgtgtctcagtcccaatgtggccgattaccctctcaggtcggctacgtatcattgccatggtgagccgttaccccaccatctagctaatacgccgcgggaccatccaaaagtgatagccgaagccatctttcaanctcggaccatgcggtccaagttgttatgcggtattagcatctgtttccaggtgttatcccccgcttctgggcaggtttcccacgtgttactcaccagttcgccactcactcaaatgtaaatcatgatgcaagcaccaatcaataccagagttcgttcgacttgcatgtattangcacgccgccagcgttcgtcctgagc

SEQ ID NO: 5 - DNA, 739 bp, Lactobacillus brevis
  misc_feature (560)..(560): n is a, t, g, or c
  misc_feature (715)..(715): n is a, t, g, or c
acagccgccttcgccactggtgttcttccatatatctacgcattccaccgctacacatggagttccactgtcctcttctgcactcaagtctcccagtttccgatgcacttctccggttaagccgaaggctttcacatcagacttaaaaaaccgcctgcgctcgctttacgcccaataaatccggacaacgcttgccacctacgtattaccgcggctgctggcacgtagttagccgtggctttctggttaaataccgtcaacccttgaacagttactctcaaaggtgttcttctttaacaacagagttttacgagccgaaacccttcttcactcacgcggcattgctccatcagactttcgtccattgtggaagattccctactgctgcctcccgtaggagtttgggccgtgtctcagtcccaatgtggccgattaccctctcaggtcggctacgtatcatcgtcttggtgggcctttacctcaccaactaactaatacgccgcgggatcatccagaagtgatagccgaagccacctttcaaacaaaatccatgcggattntgttgttatacggtattagcacctgtttccaagtgttatcccctgcttctgggcagatttcccacgtgttactcaccagttcgccactcgcttcattgttgaaatcagtgcaagcacgtcattcaacggaagctcgttcgacttgcatgtattangcatgccgccagcgttcgtcctga

SEQ ID NO: 6 - DNA, 765 bp, Lactobacillus curvatus
  misc_feature (737)..(737): n is a, t, g, or c
gcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaagaaaccgcctgcgctcgctttacgcccaataaatccggacaacgcttgccacctacgtattaccgcggctgctggcacgtagttagccgtggctttctggttggataccgtcactacctgatcagttactatcaaatacgttcttctccaacaacagagttttacgatccgaaaaccttcttcactcacgcggcgttgctccatcagactttcgtccattgtggaagattccctactgctgcctcccgtaggagtctgggccgtgtctcagtcccagtgtggccgattaccctctcaggtcggctatgcatcacggtcttggtgagcctttacctcaccaactaactaatgcaccgcgggtccatcctaaagtgatagccgaaaccatctttcaaccttgcaccatgcggtgctaggttttatgcggtattagcatctgtttccaaatgttatcccccactttagggcaggttacccacgtgttactcacccgtccgccactcactcaaatgttatcaatcagaagcaagcttcttcaatctaacgagagtgcgttcgacttgcatgtattangcacgccgccagcgttcgtcctgagcca

SEQ ID NO: 7 - DNA, 767 bp, Lactobacillus reuteri
  misc_feature (166)..(166): n is a, t, g, or c
  misc_feature (456)..(456): n is a, t, g, or c
  misc_feature (561)..(561): n is a, t, g, or c
  misc_feature (635)..(635): n is a, t, g, or c
  misc_feature (728)..(728): n is a, t, g, or c
agcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaancaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattccctactgctgcctcccgtaggagtatggaccgtgtctcagttccattgtggccgatcagnctctcaactcggctatgcatcatcgccttggtaagccgttaccttaccaactagctaatgcaccgcaggtccatcccagagtgatagccaaagccatctttcaancaaaagccatgtggcttttgttgttatgcggtattagcatctgtttccaaatgttatcccccgctccggggcangttacctacgtgttactcacccgtccgccactcactggtgatccatcgtcaatcaggtgcaagcaccatcaatcagttgggccagtgcgtacnacttgcatgtattaggcacaccgccggcgttcatcctga

SEQ ID NO: 8 - DNA, 750 bp, Lactobacillus casei
  misc_feature (31)..(31): n is a, t, g, or c
  misc_feature (34)..(34): n is a, t, g, or c
  misc_feature (37)..(37): n is a, t, g, or c
  misc_feature (687)..(687): n is a, t, g, or c
  misc_feature (717)..(717): n is a, t, g, or c
agttttggtcgatgaacggtgcttgcactgngantcnacttaaaacgagtggcggacgggtgagtaacacgtgggtaacctgcccttaagtgggggataacatttggaaacagatgctaataccgcataaatccaagaaccgcatggttcttggctgaaagatggcgyaagctatcgcttttggatggacccgcggcgtattagctagttggtgaggtaacggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaangcggctgtctggtctgtaactgacgctgangctcgaaagcatgggtagcgaacaggattagat

SEQ ID NO: 9 - DNA, 754 bp, Lactobacillus fermentum
  misc_feature (26)..(26): n is a, t, g, or c
  misc_feature (235)..(235): n is a, t, g, or c
  misc_feature (701)..(701): n is a, t, g, or c
tcgacgcgttggcccaattgattganggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaanggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaangcggctacctggtctgcaactgacgctgagactcgaaagcatgggtagcgaac

SEQ ID NO: 10 - DNA, 840 bp, Lactobacillus brevis
aaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagtcgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtca